(19) United States
(12) Patent Application Publication
(10) Pub. No.: US 2007/0060900 A1
(43) Pub. Date: Mar. 15, 2007

(54) Ostomy Device with Deodorizing Efficacy

(75) Inventors: Gerald L. Maurer, Cincinnati, OH (US); Elizabeth I. Maurer, Cincinnati, OH (US)

Correspondence Address: LAFKAS PATENT LLC, 7811 LAUREL AVENUE, CINCINNATI, OH, 45243, US

(73) Assignee: NATIONAL RESEARCH LABORATORIES, LTD., Cincinnati, OH (US)

(21) Appl. No.: 11/469,083

(22) Filed: Aug. 31, 2006

Related U.S. Application Data
(60) Provisional application No. 60/596,111, filed on Sep. 1, 2005.

(51) Int. Cl. A61F 5/44

(52) U.S. Cl. 604/333

(57) ABSTRACT

A method of reducing odor from ostomy devices by using a hydrated dialkali monometal polycarboxylate 1:1 molar ratio of metal-to-complexing agent suspended in a carrier. As the ostomy device is filled with waste products, the hydrated dialkali monometal polycarboxylate 1:1 molar ratio of metal-to-complexing agent oxidizes the molecules of the waste products and neutralizes odor-causing molecules, and thereby reduces associated odors.

### CROSS-REFERENCE TO RELATED APPLICATION

This application for a patent claims priority to U.S. Provisional Patent Application No. 60/596,111 as filed Sep. 1, 2005.

### BACKGROUND

The present invention relates to ostomy devices. More particularly, the present invention relates to ostomy devices having deodorizing efficacy as the ostomy device is used.

As a part of treatment for some types of cancers, including cancer of the colon or rectum, as well as other diseases, it is sometimes medically necessary for a patient to undergo an enterostomy. Enterostomies are performed in order to create a new opening, i.e., a stoma, for the passage of metabolic wastes when normal intestinal or urinary tract functioning is interrupted or when diseases of the intestines or urinary tract cannot be treated by medications or less radical surgery.

Ostomy devices are well known in the art, and generally include a body member that is attached to an individual's abdomen and a receiving member or pouch attached to the body member. The receiving member receives the waste products from the individual's body. The body member typically adheres to the individual's body by means of an adhesive wafer having an inlet opening for accommodating a stoma or by means of a urinary tract catheter.

Improvements have been made over the years to improve the efficiency, shape, weight, inconspicuousness, and general wearability of ostomy devices. Nonetheless, many individuals, particularly those new to having an ostomy device, have concerns about leading life having an ostomy device consistent with life without an ostomy device.

One of the more significant fears and concerns of individuals with ostomy devices is worry that the device will leak and/or that odor will escape and cause embarrassment to the individual and possible unpleasantness.

In order to be sensed by an individual, molecules related to odor-causing materials must be capable of traveling through the air to activate the olfactory senses of an individual. Such odor-causing materials themselves may be solid, liquid, or gaseous. Odor-causing materials can originate from both organic and inorganic sources. Some examples of common odor-causing materials in the environment are urine, feces, food waste, and bilge water.

Most odor-causing materials generally contain similar or common types of odor-causing molecules, such as, for example, skatoles, indoles, dimethyldisulfide, amines, and ammonia. Nature typically eliminates odors caused by these and other chemicals by slowly combining the molecules that create odors with oxygen from the air. This process is called oxidation.

Prior art compositions for “odor control” rely heavily upon “odor-masking,” rather than actually controlling or modifying the odor-causing chemical. Typical odor masking utilizes strong, often overbearing, and even unpleasant perfumes or “masking agents.” “Masking agents” are generally perfumes that simply, in a very temporary fashion, distract the olfactory sense from one unpleasant odor by means of a second, less offensive odor. When such prior art compositions are used, the sense of smell is affected.

Various mechanical types of ways to limit odor from ostomy devices have been proposed, including venting and charcoal-filled covers for the ostomy pouches. However, each of these may add additional parts and increase the bulkiness and discomfort related to the ostomy devices.

Thus, what is desired is a means of deodorizing ostomy existing devices without adding additional mechanical parts or bulkiness or without merely “masking” an odor.

### SUMMARY

The various exemplary embodiments of the present invention include a method of substantially deodorizing an ostomy device having a receiving member with an inside portion and an outside portion. The method includes suspending a hydrated dialkali monometal polycarboxylate 1:1 molar ratio of metal-to-complexing agent with a carrier, and applying the carrier with the agent to the inside portion of the receiving member.

The various exemplary embodiments of the present invention further include an ostomy device comprised of a receiving member having an inside portion and an outside portion, wherein a carrier is applied to the inside portion and dried such that the carrier comprises a substrate combined with a hydrated dialkali monometal polycarboxylate 1:1 molar ratio of metal-to-complexing agent.

### DETAILED DESCRIPTION

The compositions of this invention are designed to react chemically with the odor-causing agent at its source; thereby eliminating the cause of the odor rather than simply masking the effects of the odor. As opposed to the prior art methods in which odors are masked, the present compositions substantially neutralize actual causes of odors.

The metal complexes of the present compositions achieve the same results as natural oxidation, but at a greatly increased rate of speed, e.g., within minutes or even seconds. The present compositions comprise a metal-to-complexing agent and a polyfunctional organic ligand in a ratio of 1:1 of the metal to the ligand and has a dissociation property represented by a sigmoidally shaped plot on a pM-pH diagram.

Specific examples of the metal complex are dialkali metal monocopper(II) citrates represented by disodium-, dipotassium- or dilithiummonocopper(II) citrate. These dialkali monocopper(II) citrates have a dissociation property represented by a sigmoidal plot, wherein the curve of two directions meet at a point within the pH range of about 7 to about 9. It has been established that these monocopper(II) complexes in basic media, on the order of about pH 9 to about 12, are very stable, i.e., have an effective stability constant, K_{eff}, of the order of about 10¹² to about 10¹³. However, K_{eff} of these monocopper(II) citrate complexes at a pH of about 7-9 are on the order of about 10⁵ to about 10¹². Therefore, at a pH of around 7, the effective stability constant of the monocopper(II) citrate complex is considerably lower (a thousand to a several hundreds of thousand times lower) and a significant free Cu⁺⁺ concentration is available for anti-inflammatory activity. For example, about 10% of the copper in the complex is in the ionized state at or about pH 7 while approximately 0.1% of the copper is ionized at or about pH 9.

One especially successful metal complex for increasing such oxidation is in the 1:1 dialkali monometal polyfunctional organic ligand chelate family, exemplified in disodium monocopper (11) citrate dihydrate, CAS Registry #65330-59-8. This is sold under the name MCC™ by Chemical Systems International, Inc. of Cincinnati, Ohio.

Films, typically plastics, comprising the receiving members of ostomy devices, often have fair odor barrier properties. It is desired that such films produce minimal noise when flexed or wrinkled to avoid embarrassment to users. Typically, films currently in use for ostomy devices utilize polyvinylidene chloride (PVDC) or copolymers of vinylidene chloride with a comonomer such as methylacrylate or vinylchloride as the gas barrier layer of a multilayer film. Such films create little noise which may alert others to the presence of the ostomy device, and the films are not too bulky in nature.

In the various exemplary embodiments of the present invention, a hydrated dialkali monometal polycarboxylate 1:1 molar ratio of metal-to-complexing agent may be used in conjunction with known ostomy devices to substantially limit odors emanating from matter in ostomy devices.

Preferred hydrated dialkali monometal polycarboxylate 1:1 molar ratio of metal-to-complexing agents include disodium monocopper(II) citrate dihydrate (MCC), disodium monzinc(II) citrate (MZC), and combinations thereof. Additionally, various complexing agents other than citrate have been suitable, including phosphinates and phosphonates.

In exemplary embodiments, the hydrated dialkali monometal polycarboxylate 1:1 molar ratio of metal-to-complexing agent is suspended in a carrier and applied to the films comprising the ostomy devices. In particular, an inside portion of the receiving member of the ostomy devices are coated with the hydrated dialkali monometal polycarboxylate 1:1 molar ratio of metal-to-complexing agent suspended in a carrier. The carrier suspends the hydrated dialkali monometal polycarboxylate 1:1 molar ratio of metal-to-complexing agent in a dry state on the films and remains flexible.

In another exemplary embodiment, the immobilized 1:1 complex or mixtures thereof are applied to a solid carrier that is then affixed on the interior surface of the ostomy appliance.

As ostomy devices having the applied film of the hydrated dialkali monometal polycarboxylate 1:1 molar ratio of metal-to-complexing agent and carrier on an inside portion are filled with bodily waste products, the hydrated dialkali monometal polycarboxylate 1:1 molar ratio of metal-to-complexing agent is released from the applied film. The hydrated dialkali monometal polycarboxylate 1:1 molar ratio of metal-to-complexing agent mediates both the complexation and oxidation of the odor-causing molecules in the waste products, in addition to neutralizing odor-causing molecules, and thereby substantially limits odor.

In an exemplary embodiment, the carrier is polyvinyl alcohol (PVA).

In various exemplary embodiments, an effective amount the hydrated dialkali monometal polycarboxylate 1:1 molar ratio of metal-to-complexing agent, for example as MCC alone or with other 1:1 metal complexes, is mixed with liquid PVA which is then applied to the interior surface of the ostomy device.

In another embodiment, the PVA-based mixture is applied to a carrier, such as, for example, an adhesive backed absorbent material, dried to a film and subsequently attached to the interior of the ostomy device.

In yet another embodiment, the 1:1 metal complex or mixtures thereof are applied to an adhesive-backed absorbent material such as a cellulosic fabric or a suitable plastic which is capable of absorbing the 1:1 metal complex or mixtures thereof, dried to the substantially solid state and subsequently adhered to the interior of the ostomy device.

It can be seen that there are a number of ways to join the odor-reducing complexes to the interior of the of ostomy devices. The most suitable method of adhering the metal complex or complexes to the interior of the ostomy devices is determined by the manufacturing process of the particular ostomy device.

Thus, it is apparent that there has been provided a method for controlling odors associated with ostomy devices by introduction of a metal complex, in accordance with the present invention, which satisfies the aspects and objects as set forth above.

While the invention has been described in conjunction with specific embodiments, it is evident that many alternatives, modifications and variations will be apparent to those skilled in the art in light of the foregoing description and examples, and without deviating from the contemplated scope of the present invention. Accordingly, it is intended to embrace all such alternatives, modifications and variations which fall within the spirit and scope of the appended claims.

What is claimed is:

1. A method of substantially deodorizing an ostomy device having a receiving member with an inside portion and an outside portion, comprising:
suspending a hydrated dialkali monometal polycarboxylate 1:1 molar ratio of metal-to-complexing agent with a carrier; and
applying the carrier with the agent to the inside portion of the receiving member.

2. The method according to claim 1, wherein the hydrated dialkali monometal polycarboxylate 1:1 molar ratio of metal-to-complexing agent is disodium monocopper(II) citrate dihydrate (MCC).

3. The method according to claim 1, wherein the hydrated dialkali monometal polycarboxylate 1:1 molar ratio of metal-to-complexing agent is disodium monzinc(II) citrate (MZC).

4. The method according to claim 1, wherein the carrier is polyvinyl alcohol.

5. The method according to claim 1, wherein the carrier is an adhesive-backed substrate to which the agent is affixed.

6. The method according to claim 5, wherein the adhesive-backed substrate is a cellulosic fabric.

7. An ostomy device, comprising:
a receiving member having an inside portion and an outside portion, wherein a carrier is applied to the inside portion and dried such that the carrier comprises a substrate combined with a hydrated dialkali monometal polycarboxylate 1:1 molar ratio of metal-to-complexing agent.

8. The device according to claim 7, wherein the hydrated dialkali monometal polycarboxylate 1:1 molar ratio of metal-to-complexing agent is disodium monocopper(II) citrate dihydrate (MCC).

9. The device according to claim 7, wherein the hydrated dialkali monometal polycarboxylate 1:1 molar ratio of metal-to-complexing agent is disodium monzinc(II) citrate (MZC).

10. The device according to claim 7, wherein the carrier is polyvinyl alcohol.

11. The device according to claim 7, wherein the carrier is an absorbent material comprising the agent and the carrier being affixed to the inside portion of the device.
